(19) United States
(12) Patent Application Publication
(10) Pub. No.: US 2011/0275953 A1
(43) Pub. Date: Nov. 10, 2011

(54) Hearing Treatment In Patients With Questionable Cochlear Nerve Functionality

(75) Inventors: Marek Polak, Innsbruck (AT); Fransecs Roca-Ribas Serdá, Barcelona (ES); Javier Gavilán Bouzas, Madrid (ES); Luis Lassaletta Atienza, Madrid (ES); Nuria Miró Castillo, Barcelona (ES); Julio Rodrigo Dacosta, Madrid (ES)

(73) Assignee: MED-EL ELEKTROMEDIZINISCHE GERAETE GMBH, Innsbruck (AT)

(21) Appl. No.: 13/101,417

(22) Filed: May 5, 2011

Related U.S. Application Data
(60) Provisional application No. 61/331,552, filed on May 5, 2010.

(51) Int. Cl. A61B 5/05

(52) U.S. Cl. 600/554

(57) ABSTRACT

An intraoperative approach for objective determination of auditory nerve functioning is described. Following surgical removal of tumor tissue impairing auditory functioning of a patient, an electrode array having electrode contacts is intra-operatively inserted into the cochlea of the patient. A diagnostic stimulation signal is provided to the electrode contacts to stimulate auditory nerve tissue within the cochlea. And an electrically evoked functional response is measured of the auditory nerve tissue to the stimulation signal.

This application claims priority from U.S. Provisional Patent Application 61/331,552, filed May 5, 2010, which is incorporated herein by reference.

### FIELD OF THE INVENTION

The present invention relates to auditory prostheses, and more particularly, an objective measurement arrangement related to providing patient treatment with such systems.

### BACKGROUND ART

Patients with certain types of tumors such as NF2, acoustic neurinoma, vestibular schawanoma or meningioma often lose their hearing due to expansion of the tumor. Patients diagnosed with such tumors then undergo tumor removal surgery. Depending on the size and location of the tumor, the patient's auditory nerve (CNVIII—the VIIIth Cranial Nerve) often cannot be preserved. Usually, these patients experience sudden hearing loss prior to the surgery, typically with an acoustic response such that evoked potentials don't show any response to acoustic stimuli.

Auditory function performance after having loss of the auditory nerve in NF2 subjects was shown in Behr et al., Experiences And Results With Auditory Brainstem Implants (ABI) In NF-2 Patients, Clinical Neurophysiology April 2007 (Vol. 118, Issue 4, Page e14); Schwartz et al., Auditory Brainstem Implants, Neurotherapeutics, 5(1): pp. 128-36, 2008; both of which are incorporated herein by reference. There is additional literature describing patients suffering from acoustic neurinoma who received a cochlear implant (CI) when the acoustic nerve was not physically removed, where some patients responded as well as any other CI patient, while others did not respond at all immediately after the initial stimulation (Vincenti V., Hearing Rehabilitation in Neurofibromatosis Type 2 Patients: Cochlear versus Auditory Brainstem Implantation, Audiology & Neurotology 2008, Vol. 13, No. 4; incorporated herein by reference).

Over the last two decades, hearing rehabilitation in post-surgical totally deaf NF2 patients has improved from no chance at all, into the possibility to choose (at least in some cases) between two different kinds of implant systems—an auditory brainstem implant (ABI) or a cochlear implant (CI). If the auditory nerve is not preserved after the surgery, then an ABI should be implanted. But if the auditory nerve is preserved after the tumor removal surgery, then either an ABI or a CI can be used. Patients implanted with a CI usually do significantly better on speech tests than patients implanted with an ABI. Unfortunately, the physical presence of the acoustic nerve does not always indicate remaining auditory functionality.

Until now, the decision to use a CI for post-surgical hearing treatment has been based either on no function measurements at all, or at best, post-operative subjective or objective measurements. Where no function measurements were performed, surgeons relied on and intra-operative visual judgment of auditory nerve preservation and/or post-operative imaging techniques and outcomes.

There is another group of patients in which it is not known whether CI implantation is the proper choice of treatment for their hearing loss. This group includes patients with narrow auditory canal and/or cochlear hypoplasia. For example, for many patients with a narrow auditory canal, presence of the auditory nerve cannot be determined using conventional imaging techniques. This makes it necessary to perform an objective test of the functionality of the acoustic nerve. Unfortunately, pre-operative objective measures cannot reliably verify the functionality of the cochlear nerve. Currently, a pre-operative electrically evoked electrocochleaography (ECochG) of the round window niche or an auditory brainstem measurement is performed before deciding as to cochlear implantation.

Although this measurement can help in many cases to decide whether a patient would benefit from CI or ABI, it may not be sufficient in all cases to judge whether or not a CI would be beneficial. For example, see Kileny and Zwolan, Pre-Perioperative, Transtympanic Electrically Evoked Auditory Brainstem Response in Children, Int. J. Audio 1. 43 Suppl 1 2004, p. S16-S21; McMahon et al., Frequency-Specific Electrocochleography Indicates That Presynaptic And Postsynaptic Mechanisms Of Auditory Neuropathy Exist, Ear Hear. 29(3), 2008, p. 314-325; both incorporated herein by reference. In McMahon, subjects diagnosed with auditory neuropathy benefited from a proposed hearing treatment method wherein the auditory neuropathy was on the basis of absent ABR and large CM waveforms (measured with round-window ECochG). The measuring system can be used as a hearing preservation monitoring system during the skull base surgery that affects the cochlear nerve. The measuring electrode can be placed in the cochlea and the changes in evoked potentials recorded during every surgical step. Any clear changes in the latencies and amplitudes that are noted may imply damage to the nerve.

### SUMMARY OF THE INVENTION

Embodiments of the present invention are directed to an intraoperative approach for objective determination of auditory nerve functioning. For example, in the case of tumor patients, following surgical removal of tumor tissue impairing auditory functioning, an electrode array having electrode contacts is intra-operatively inserted into the cochlea of the patient. A diagnostic stimulation signal is provided to the electrode contacts to stimulate auditory nerve tissue within the cochlea. And an electrically evoked functional response is measured of the auditory nerve tissue to the stimulation signal.

Selecting the post-operative auditory treatment may include selecting an appropriate type of auditory implant system, e.g., selecting between a cochlear implant (CI) system and an auditory brainstem implant (ABI) system. The tumor tissue may include any or all of Neurofibromatosis 2 (NF2), acoustic neurinoma, vestibular schawanoma, and/or meningioma tumor tissue.

Inserting the electrode array may include placing a separate reference electrode, either within the cochlea or outside the cochlea. Measuring the electrically evoked functional response may specifically include measuring frequency-specific characteristics of auditory nerve fiber, measuring an electrically evoked auditory brainstem response (EABR) and/or measuring an electrically evoked action potential (ECAP) response.

Embodiments of the present invention also include an intraoperative system for objective determination of auditory nerve functioning. An electrode array having a plurality of electrode contacts is intra-operatively inserted into the cochlea of a patient having questionable cochlear nerve functionality. There is means for providing a diagnostic stimulation signal to the electrode contacts to stimulate auditory nerve tissue within the cochlea. Then, an electrically evoked functional response of the auditory nerve tissue to the stimulation signal is measured. From this, the functionality of the cochlear nerve and a corresponding appropriate therapeutic treatment are determined.

In such embodiments, the corresponding appropriate therapeutic treatment may include an appropriate type of auditory implant system. Inserting the electrode array may include placing a separate reference electrode, for example, placing the reference electrode within the cochlea or outside the cochlea. Measuring the electrically evoked functional response may include measuring frequency-specific characteristics of auditory nerve fiber, measuring an electrically evoked auditory brainstem response (EABR), and/or measuring an electrically evoked action potential (ECAP) response. The questionable cochlear nerve functionality may be related to tumor tissue impairing auditory functioning of the patient, a narrow auditory canal, and/or a non-tumor auditory neuropathy.

### BRIEF DESCRIPTION OF THE DRAWINGS

FIG. 1 shows a block diagram of a measurement system according to an embodiment.

FIG. 2 shows an example of an electrode array used for intraoperative measurements according to an embodiment of the present invention.

FIG. 3 show an example of EABR recordings taken after removal of an acoustic neurinoma.

### DETAILED DESCRIPTION OF SPECIFIC EMBODIMENTS

Published literature in the field reports on results with patients following tumor removal surgery for whom the auditory nerve was preserved. Some such patients received cochlear implants directly without any prior tests, while others received cochlear implants later, after the surgical incision had healed and following electrical stimulation measurement of the round window or promontory. The patients were asked if they heard any acoustic sensation after applying the electrical stimuli—but other literature suggests that such sensations do not always result from acoustic stimuli and the sensations could be tactile.

Applicants believe that an objective measurement would be more appropriate, and embodiments of the present invention are based upon an objective measurement which is performed intra-surgically after tumor removal to determine whether or not the auditory nerve remains functioning. With that information, the surgeon can then immediately decide what type of post-operative treatment is optimally indicated—e.g. what specific type of auditory implant system should be used. Thus, following surgical removal of tumor tissue impairing auditory functioning of a patient, an electrode array having electrode contacts is intra-operatively inserted into the cochlea of the patient. A diagnostic stimulation signal is provided to the electrode contacts to stimulate auditory nerve tissue. And an electrically evoked functional response is measured of the auditory nerve tissue to the stimulation signal.

Usually, the most critical part of tumor removal is removing tissue from within the internal auditory canal. Experienced surgeons can say very reliably whether or not all of the tumor was removed, and they also reliably know whether or not the auditory nerve was preserved. An objective intra-operative measurement approach as in embodiments of the present invention allows for an instantaneous decision during surgery for removal of tumors affecting the auditory system of an appropriate post-surgical hearing treatment—that is, either implantation with either a CI or an ABI, whichever type of prosthetic device the objective measurement information (e.g., frequency specific information regarding the functionality of auditory nerve fibers) indicates will most benefit the patient. This allows the surgeon to know much more reliably whether to open the CI package or the ABI package. This is extremely useful information because patients may receive immediately an appropriate choice of treatment and thus benefit from their hearing sooner. In addition, it is known that electrical stimulation decreases the further degeneration of the auditory path so that the faster benefit from the hearing treatment also increases the patient's ability to hear. Furthermore, once a package has been opened, the device within the package either has to be implanted immediately or be disposed—later usage of a device from an open package is strictly forbidden for reasons of sterility. Moreover, implanting the correct treatment device during the tumor removal surgery avoids the need for a second additional surgery for implantation of the prosthetic device for hearing treatment. And for non-tumor patients, patients with narrow auditory canal, or patients suffering from auditory neuropathy (as defined in McMahon et al., 2008), the intraoperative measurement outcomes also imply the advantages as above.

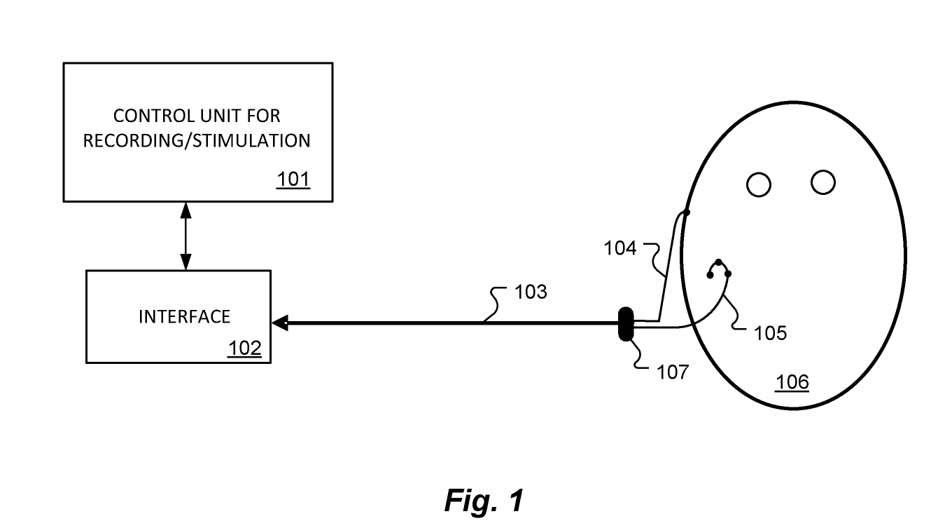

FIG. 1 shows a block diagram of a measurement system according to one specific embodiment which can perform either or both of an electrically Evoked Auditory Brain stem Responses (EABR) and/or an electrically evoked compound action potential (ECAP) measurements and recordings. Control Unit 101 for Recording and Stimulation, for example, a Med-El Maestro CI system, generates stimulation signals and analyzes response measurements. Connected to the Control Unit 101 is an Interface Box 102, for example, a Diagnostic Interface System such as the DIB II conventionally used with the Maestro system and/or an ABI stimulation box that formats and distributes the input and output signals between the Control Unit 101 and the system components implanted in the patient 106. For example, as shown in FIG. 1, there may be an Interface Lead 103 connected at one end to the Interface Box 102 and at the other end having Electrode Plug 107 that then divides into an Extra-Cochlear Branch 104 and an Intra-Cochlear Branch 105.

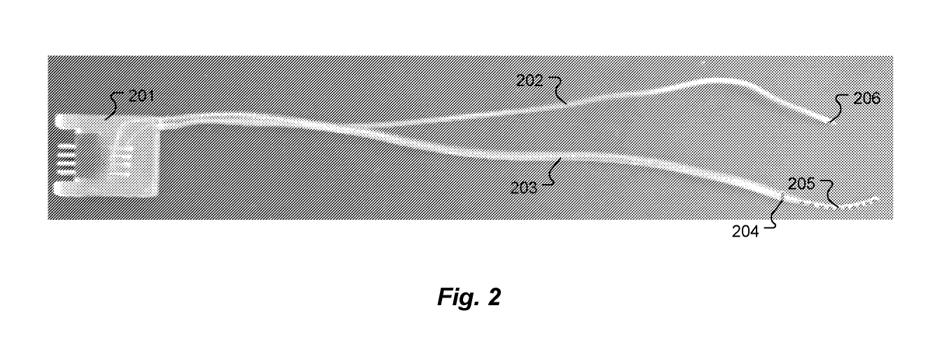

FIG. 2 shows a photograph of one specific example of the electrode components that are implanted for intraoperative measurements according to an embodiment of the present invention. Electrode Plug 201 can be connected to an Interface Lead 103 or directly to the Interface Box 102. Intra-Cochlear Branch 203 is gently inserted by the surgeon into the cochlea up to an Insertion Mark 204, e.g., ˜16 mm from the distal tip of the Intra-Cochlear Branch 203. Intra-Cochlear Branch 203 also has one or more Intra-Cochlear Electrode Contacts 205 (e.g., three) towards the distal end for applying stimulation signals to and/or taking measurements from the adjacent audio nerve tissue. Extra-Cochlear Branch 202 and Extra-Cochlear Electrode Contact 206 act as a reference electrode for the system, for example, typically placed in muscle temporalis. In some embodiments, there may be a reference electrode on the Intra-Cochlear Branch 203, in which case, an Extra-Cochlear Branch 202 may not be needed.

After the tumor is removed, the Intra-Cochlear Electrode Branch 203 would be implanted into the cochlea, which should be less traumatic than for a regular cochlear implant array. By stimulating different Intra-Cochlear Electrode Contacts 205 in the Intra-Cochlear Electrode Branch 203 frequency specific information can be collected for the functionality of the auditory nerve fibers. Measurements and recordings should be evaluated for more than one Intra-Cochlear Electrode Contacts 205 to confirm preservation of cochlear nerve fibers in different locations within the cochlea. For that purpose, separation of the Intra-Cochlear Electrode Contacts 205 is typically around about 4 mm. If the objectively measured response is favorable, that reliably indicates that at least some cochlear nerve fibers have been preserved and the auditory nerve is functioning.

By definition, the CAP is an alternating current response which is generated by the cochlear end of the VIIIth Cranial Nerve, and it represents the summed response of the synchronous firing of thousands of auditory nerve fibers (see, e.g., Ferraro J, LaVar G. Best and I. Kaufman Arenberg; The Use of Electrocochleography in the Diagnosis, Assessment, and Monitoring of Endolymphatic Hydrops, Otolaryngologic Clinics of North America; 16:1, pp. 69-82; February, 1983; Hall J W., Handbook of Auditory Evoked Responses, Allyn and Bacon; Needham Heights, Mass., 1992; both of which are incorporated herein by reference). Auditory brainstem response (ABR) is an electrical signal evoked from the brainstem of a human or other mammal by the presentation of a specific signal (see, e.g., Jewett D L, Romano M N, Williston J S, Human Auditory Evoked Potentials: Possible Brain Stem Components Detected On The Scalp, Science 167:1517-8; 1970; Katz J., Handbook of Clinical Audiology, 4th Edition, 1994; both of which are incorporated herein by reference). The relationship of specific wave components of the ABR to the components of the auditory pathway can be represented as follows: wave I: Cochlear Action Potential (CAP), distal CNVIII; wave II: proximal CNVIII; wave III: Cochlear Nuclei; wave IV: Superior Olivary Complex; and wave V: Lateral Lemniscus. The definition of peaks is according to Jewett D L, Williston J S, Auditory-Evoked Far Fields Averaged From The Scalp Of Humans, Brain 94(4):681-96; 1971; incorporated herein by reference.

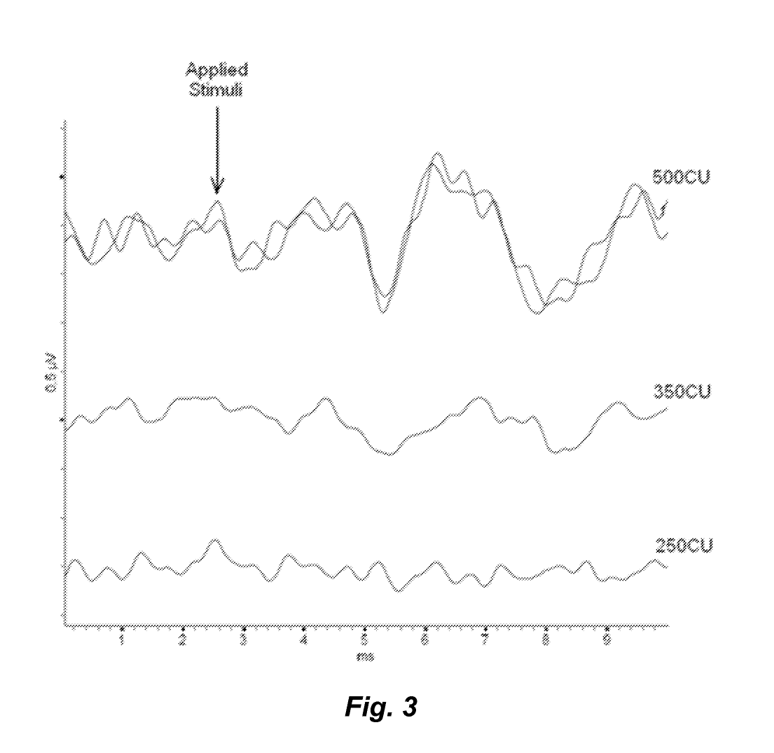

FIG. 3 shows an example of EABR recordings taken after removal of an acoustic neurinoma. The recording electrode was obtained from the medial electrode of a sample electrode array such as the one shown in FIG. 2. After obtaining this specific recording, the NF2 patient in question received a cochlear implant.

Embodiments of the invention may be implemented in whole or in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments can be implemented in whole or in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. An intraoperative system for objective determination of auditory nerve functioning, the system comprising:
means for intra-operatively inserting into the cochlea of a patient an electrode array having a plurality of electrode contacts following surgical removal of tumor tissue impairing auditory functioning of the patient;
means for providing a diagnostic stimulation signal to the electrode contacts to stimulate auditory nerve tissue within the cochlea; and
means for measuring an electrically evoked functional response of the auditory nerve tissue to the stimulation signal.

2. A system according to claim 1, further comprising:
means for selecting a post-operative auditory treatment for the patient based on the measured functional response.

3. A system according to claim 2, wherein the means for selecting the post-operative auditory treatment includes means for selecting an appropriate type of auditory implant system.

4. A system according to claim 1, wherein means for inserting the electrode array includes means for placing a separate reference electrode.

5. A system according to claim 4, wherein the means for placing the reference electrode place the reference electrode within the cochlea.

6. A system according to claim 4, wherein the means for placing the reference electrode places the reference electrode outside the cochlea.

7. A system according to claim 1, wherein the means for measuring the electrically evoked functional response includes means for measuring frequency-specific characteristics of auditory nerve fiber.

8. A system according to claim 1, wherein the means for measuring the electrically evoked functional response includes means for measuring an electrically evoked auditory brainstem response (EABR).

9. A system according to claim 1, wherein the means for measuring the electrically evoked functional response includes means for measuring an electrically evoked action potential (ECAP) response.

10. A system according to claim 1, wherein the tumor tissue includes Neurofibromatosis 2 (NF2) tumor tissue.

11. A system according to claim 1, wherein the tumor tissue includes acoustic neurinoma tumor tissue.

12. A system according to claim 1, wherein the tumor tissue includes vestibular schawanoma tumor tissue.

13. A system according to claim 1, wherein the tumor tissue includes meningioma tumor tissue.

14. An intraoperative system for objective determination of auditory nerve functioning, the system comprising:
means for intra-operatively inserting an electrode array having a plurality of electrode contacts into the cochlea of a patient having questionable cochlear nerve functionality;
means for providing a diagnostic stimulation signal to the electrode contacts to stimulate auditory nerve tissue within the cochlea;
means for measuring an electrically evoked functional response of the auditory nerve tissue to the stimulation signal; and
means for determining functionality of the cochlear nerve and a corresponding appropriate therapeutic treatment.

15. A system according to claim 14, wherein the corresponding appropriate therapeutic treatment includes an appropriate type of auditory implant system.

16. A system according to claim 14, wherein means for inserting the electrode array includes means for placing a separate reference electrode.

17. A system according to claim 16, wherein the means for placing the reference electrode place the reference electrode within the cochlea.

18. A system according to claim 16, wherein the means for placing the reference electrode places the reference electrode outside the cochlea.

19. A system according to claim 14, wherein the means for measuring the electrically evoked functional response includes means for measuring frequency-specific characteristics of auditory nerve fiber.

20. A system according to claim 14, wherein the means for measuring the electrically evoked functional response includes means for measuring an electrically evoked auditory brainstem response (EABR).

21. A system according to claim 14, wherein the means for measuring the electrically evoked functional response includes means for measuring an electrically evoked action potential (ECAP) response.

22. A system according to claim 14, wherein the questionable cochlear nerve functionality is related to tumor tissue impairing auditory functioning of the patient.

23. A system according to claim 14, wherein the questionable cochlear nerve functionality is related to a narrow auditory canal.

24. A system according to claim 14, wherein the questionable cochlear nerve functionality is related to a non-tumor auditory neuropathy.
